(19) United States
(12) Patent Application Publication
(10) Pub. No.: US 2024/0233941 A9
(43) Pub. Date: Jul. 11, 2024

(54) MEDICAL DIAGNOSIS SUPPORT DEVICE, MEDICAL DIAGNOSIS SUPPORT METHOD, AND STORAGE MEDIUM

(71) Applicant: CANON MEDICAL SYSTEMS CORPORATION, Otawara-shi (JP)

(72) Inventors: Sho SASAKI, Utsunomiya (JP); Kazumasa NORO, Shioya (JP); Kosuke ARITA, Nasushiobara (JP)

(73) Assignee: CANON MEDICAL SYSTEMS CORPORATION, Otawara-shi (JP)

(21) Appl. No.: 18/489,171

(22) Filed: Oct. 18, 2023

(30) Foreign Application Priority Data
Oct. 20, 2022 (JP) 2022-168228

(51) Int. Cl. G16H 50/20; G16H 50/70; G16H 70/60

(52) U.S. Cl. CPC G16H 50/20; G16H 50/70; G16H 70/60

(57) ABSTRACT

A medical diagnosis support device of an embodiment includes processing circuitry. The processing circuitry acquires first reference data based on first clinical data relating to a diagnosis result of an evaluation target and second reference data based on second clinical data relating to a comparison target diagnosis result for the evaluation target and performs an analysis process relating to the diagnosis result of the evaluation target based on the first reference data and the second reference data. The first reference data includes first data that has been referred to by a first user to obtain the diagnosis result of the evaluation target and second data that has been referred to by a diagnosis support system to obtain the diagnosis result of the evaluation target. The second reference data includes the first data that has been referred to by at least one of the first user and a second user different from the first user in order to obtain the diagnosis result of the comparison target.

### CROSS-REFERENCE TO RELATED APPLICATION

The present application claims priority based on Japanese Patent Application No. 2022-168228, filed Oct. 20, 2022, the content of which is incorporated herein by reference.

### FIELD

Embodiments disclosed in the present specification and drawings relate to a medical diagnosis support device, a medical diagnosis support method, and a storage medium.

### BACKGROUND

Conventionally, technology relating to a medical diagnosis system that determines whether or not a diagnosis has been appropriately performed on the basis of the presence or absence of reference of a result of a patient examining process of a physician or a reference period of time has been proposed. However, because the conventional medical diagnosis system determines whether or not a diagnosis has been appropriately performed in a process of comparing features decided on by a physician who performs the diagnosis in advance, it is difficult to determine whether or not the diagnosis corresponding to an individual case has been performed. Furthermore, in conventional medical diagnosis systems, it is difficult to determine whether or not a physician is performing an appropriate clinical process (examination) when diagnosing a patient.

In recent years, the study and introduction of clinical decision support (CDS) systems incorporating machine learning functions have been promoted. The clinical decision support system is useful for obtaining correct diagnosis results for patients by supporting a diagnosis process of a physician. As a machine learning function incorporated in the clinical decision support system, for example, it is conceivable to use a trained model trained by artificial intelligence (AI). However, in this case, there is a possibility that biases (for example, prejudice, unfairness, and the like) will be included in the diagnosis results due to AI-trained models, machine learning algorithms, and the like. Therefore, a process of removing (eliminating) biases caused by a trained model and a machine learning algorithm, for example, by using fairness-aware machine learning as the machine learning function incorporated in the clinical decision support system, is conceivable. In this case, a process in which the clinical decision support system can correctly support the physician and provide guidance for the diagnosis result for the patient in the correct direction is conceivable.

However, if the clinical decision support system provides the guidance for the diagnosis results in the correct direction due to the incorporation of fairness-aware machine learning, for example, the physician's diagnosis will focus on the diagnosis result of the clinical decision support system (the physician will rely on the diagnosis result of the clinical decision support system), and the physician may not be able to make the correct diagnosis. In other words, the diagnosis result of the physician using a clinical decision support system that incorporates fairness-aware machine learning may not necessarily be a correct diagnostic result. This is because it is considered that a procedure performed to obtain a diagnosis result such as correctly confirming the patient's examination results must also be taken into account until the physician derives the correct diagnosis result.

### BRIEF DESCRIPTION OF THE DRAWINGS

FIG. 1 is a diagram showing an example of a functional configuration and a usage environment of a medical diagnosis support device according to an embodiment.

FIG. 2 is a diagram showing an example of information of reference data acquired by the medical diagnosis support device according to the embodiment.

FIG. 3 is a diagram schematically showing an example of a process in which the medical diagnosis support device according to the embodiment analyzes diagnosis results.

FIG. 4 is a diagram schematically showing another example of a process in which the medical diagnosis support device according to the embodiment analyzes diagnosis results.

FIG. 5 is a diagram showing an example of a method in which the medical diagnosis support device according to the embodiment provides a notification of a result of analyzing the diagnosis results.

FIG. 6 is a flowchart showing an example of a flow of a process of the medical diagnosis support device according to the embodiment.

### DETAILED DESCRIPTION

A medical diagnosis support device of an embodiment includes processing circuitry. The processing circuitry acquires first reference data based on first clinical data relating to a diagnosis result of an evaluation target and second reference data based on second clinical data relating to a comparison target diagnosis result for the evaluation target and performs an analysis process relating to the diagnosis result of the evaluation target based on the first reference data and the second reference data. The first reference data includes first data that has been referred to by a first user to obtain the diagnosis result of the evaluation target and second data that has been referred to by a diagnosis support system to obtain the diagnosis result of the evaluation target. The second reference data includes the first data that has been referred to by at least one of the first user and a second user different from the first user in order to obtain the diagnosis result of the comparison target.

Hereinafter, a medical diagnosis support device, a medical diagnosis support method, and a storage medium of embodiments will be described with reference to the drawings. The medical diagnosis support device is, for example, a device for supporting a diagnosis process of the physician so that a consistent diagnosis result is obtained while maintaining a diagnosis procedure of a physician who examines a patient in medical institutions where a clinical decision support (CDS) system incorporating machine learning functions has been introduced. For example, a medical diagnosis support device supports a patient diagnosing process of a physician with reference to information stored in a server device or a storage device incorporated in a network of a medical institution, a cloud computing system, or the like.

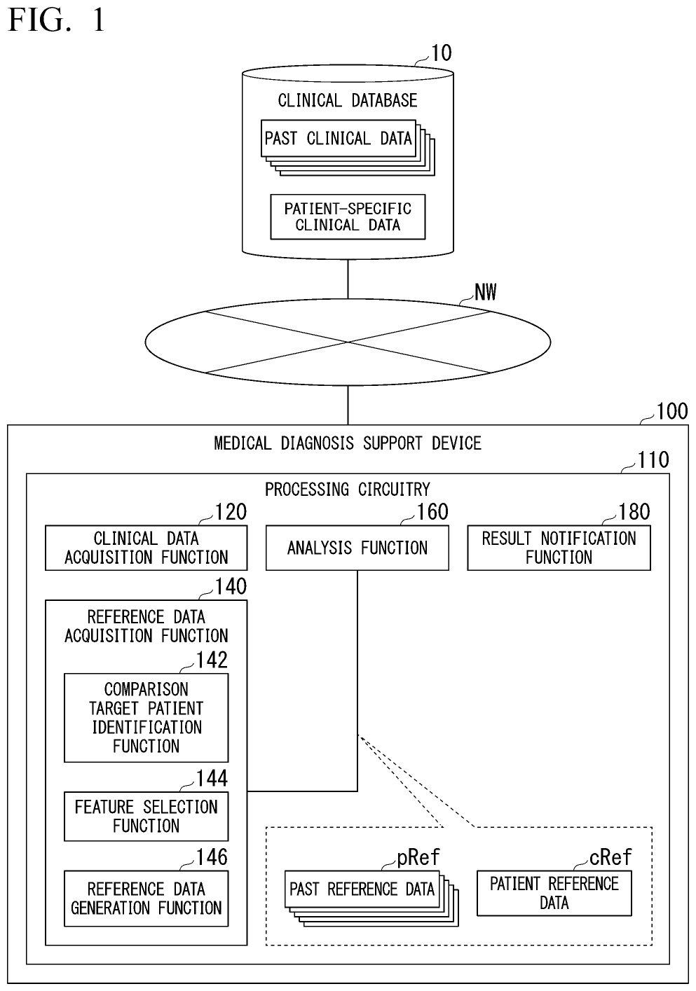

FIG. 1 is a diagram showing an example of a functional configuration and a usage environment of a medical diagnosis support device according to an embodiment. A medical diagnosis support device 100 communicates with, for example, a clinical database 10, which is a storage device storing various information relating to a clinical process for a patient, via a network NW. The network includes, for example, the Internet, a wide area network (WAN), a local area network (LAN), a provider device, a radio base station, and the like. The medical diagnosis support device 100 may be implemented by a server device or the like on a network.

The clinical database 10 stores information (data) relating to a result of diagnosis performed for the patient. The clinical database 10 is, for example, a storage device for an electronic chart system, a storage device for storing data such as patient examination results, patient examination images, and medical interview results obtained by asking a patient about his or her condition. In FIG. 1, for example, an example in which data such as past clinical data and patient-specific clinical data is stored in the clinical database 10 is shown. The past clinical data is data relating to diagnosis results of previously diagnosed patients. Past clinical data includes data relating to diagnosis results of diagnosing patients before the clinical decision support system was introduced in medical institutions and data relating to diagnosis results of diagnosing patients after the clinical decision support system was introduced. The patient-specific clinical data is data relating to the diagnosis result of the current diagnosis target patient. In the past clinical data and patient-specific clinical data, each patient's diagnosis results, data referred to by the physician to obtain the diagnosis results, and the like are associated together. Past clinical data and patient-specific clinical data after the introduction of the clinical decision support system in medical institutions are also associated with data referred to by the clinical decision support system's function of diagnosing patients using a diagnosis support model and the like. The diagnosis support model is an example of a “diagnosis support system.” Diagnosis support system is typically those that are built using trained artificial intelligence (AI) models, but are also systems that use or combine trained AI models with either other auxiliary programs or programs that perform inference.

More specifically, diagnosis results of diagnosing patients other than the current diagnosis target patient (hereinafter referred to as a “diagnosis target patient”), various data referred to by physicians (including a primary care physician performing the current diagnosis and other physicians) to obtain the diagnosis results, and the like are associated in the past clinical data. Various data referred to according to the function of the clinical decision support system and the like are also associated with the past clinical data from after the clinical decision support system was introduced in the medical institution. The past clinical data may include data associated with various data relating to diseases that the diagnosis target patient previously suffered from. In this case, the past clinical data of the diagnosis target patient is treated as past clinical data of other patients. In order to facilitate the following description, it is assumed that the past clinical data relating to the disease that the diagnosis target patient previously suffered from is the past clinical data of other patients. A primary care physician is an example of a “first user” and another physician is an example of a “second user.”

In the patient-specific clinical data, the diagnosis result of the current diagnosis of the diagnosis target patient, various data referred to by the primary care physician to obtain the diagnosis result, and the like are associated. Various data referred to by the function of the clinical decision support system and the like are also associated with the patient-specific clinical data from after the clinical decision support system was introduced in the medical institution.

Various data referred to by the physician and the function of the clinical decision support system include, for example, data (information) such as patient examination results before diagnosis, patient examination images, and medical interview results obtained by asking a patient about his or her condition. Various data referred to by the physician and the function of the clinical decision support system may include a plurality of examination results, examination images, and medical interview results with respect to one examination item or one medical interview item. If an examination or medical interview was not performed during the diagnosis, the examination result, examination image, or medical interview result may not be included.

In the following description, the data referred to by the function of the clinical decision support system in the past clinical data and the patient-specific clinical data, i.e., the data used as the input or output of the diagnosis support model in the function of the clinical decision support system, is referred to as data referred to by the diagnosis support model.

Although an example in which the past clinical data and the patient-specific clinical data are stored in the clinical database 10 is shown in FIG. 1, the past clinical data and the patient-specific clinical data may be stored in separate storage devices (databases). That is, past clinical data relating to a patient whose final diagnosis result has been confirmed and patient-specific clinical data relating to a patient whose final diagnosis result has not been confirmed (currently being diagnosed) may be stored separately.

A diagnosis target patient is an example of an “evaluation target.” The past clinical data is an example of “second clinical data” and the patient-specific clinical data is an example of “first clinical data.” The data referred to by the physician is an example of “first data” and the data referred to by the diagnosis support model is an example of “second data.”

### [Configuration of Medical Diagnosis Support Device]

The medical diagnosis support device 100 includes, for example, processing circuitry 110. The processing circuitry 110 executes, for example, a clinical data acquisition function 120, a reference data acquisition function 140, an analysis function 160, a result notification function 180, and the like. The reference data acquisition function 140 is performed to execute, for example, processes of a comparison target patient identification function 142, a feature selection function 144, a reference data generation function 146, and the like. The processing circuitry 110 implements these functions with a hardware processor executing a program (software) stored in a memory (not shown) as an example. The memory (not shown) is implemented by, for example, a semiconductor memory element such as a read-only memory (ROM), a random-access memory (RAM), or a flash memory, a hard disk drive (HDD), an optical disc, or the like.

The hardware processor is, for example, circuitry such as a central processing unit (CPU), a graphics processing unit (GPU), an application-specific integrated circuit (ASIC), or a programmable logic device (for example, a simple programmable logic device (SPLD), a complex programmable logic device (CPLD), or a field programmable gate array (FPGA)). Instead of storing the program in the memory (not shown), the program may be directly embedded in the circuit of the hardware processor. In this case, the hardware processor implements each function by reading and executing the program embedded in the circuitry. The hardware processor is not limited to being configured as a single circuit and may be configured as one hardware processor by combining a plurality of independent circuits to implement each function. A plurality of components may be integrated into one hardware processor to implement each function. Each function may be implemented by incorporating a plurality of components into one dedicated LSI circuit. Here, the program (software) may be stored in advance in a storage device (a storage device having a non-transitory storage medium) that constitutes a semiconductor memory device such as a ROM, a RAM, or a flash memory or a storage device such as a hard disk drive. Alternatively, the program (software) may be stored in a removable storage medium (a non-transitory storage medium) such as a DVD or CD-ROM and installed in a storage device provided in the medical diagnosis support device 100 when the storage medium is mounted in a drive device provided in the medical diagnosis support device 100. The program (software) may be downloaded in advance from another computer device via the network NW and installed in the storage device provided in the medical diagnosis support device 100. A program (software) installed in the storage device provided in the medical diagnosis support device 100 may be transferred to and executed by processing circuitry provided in the processing circuitry 110.

The clinical data acquisition function 120 acquires clinical data stored in the clinical database 10. More specifically, the clinical data acquisition function 120 acquires the past clinical data and the patient-specific clinical data of the current diagnosis target patient stored in the clinical database 10. At this time, the clinical data acquisition function 120 may acquire all past clinical data stored in the clinical database or may acquire only the past clinical data used when the analysis function 160 is performed to perform an analysis process in accordance with control from the reference data acquisition function 140. The clinical data acquisition function 120, for example, controls a communicator (not shown) to acquire clinical data. The clinical data acquisition function 120 is performed to output the acquired clinical data to the reference data acquisition function 140.

The reference data acquisition function 140 is performed to acquire (generate) reference data used when the analysis function 160 is performed to perform the analysis process on the basis of the past clinical data and the patient-specific clinical data acquired in the clinical data acquisition function 120. The reference data acquisition function 140 is performed to acquire (generate) reference data based on the past clinical data (hereinafter referred to as “past reference data pRef”) and reference data based on the patient-specific clinical data (hereinafter referred to as “patient reference data cRef”). The past reference data pRef and the patient reference data cRef are data to be compared in the analysis process of the analysis function 160 to determine whether or not the diagnosis result of the primary care physician for the diagnosis target patient indicated in the patient-specific clinical data is a consistent diagnosis result. More specifically, the past reference data pRef and the patient reference data cRef are data for comparing diagnosis results and various data referred to by a primary care physician for the diagnosis target patient indicated in the patient-specific clinical data with diagnosis results and various data referred to by other primary care physicians for other patients indicated in the past clinical data.

The reference data acquisition function 140 (which may include the clinical data acquisition function 120) is an example of an “acquirer.” The past reference data pRef is an example of “second reference data” and the patient reference data cRef is an example of “first reference data.”

The comparison target patient identification function 142 is performed to identify another patient (hereinafter referred to as a “comparison target patient”) to be compared with the diagnosis target patient indicated in the patient-specific clinical data. The comparison target patient identification function 142 is performed to identify a comparison target patient from other patients indicated in the past clinical data. For example, the comparison target patient identification function 142 decides whether or not to designate another patient indicated in the past clinical data as the comparison target patient by determining whether or not any one or more of the following conditions are satisfied. A comparison target patient is an example of a “comparison target.”
(Condition 1): The other patient is another patient previously diagnosed by the primary care physician for the diagnosis target patient.
(Condition 2): The other patient is another patient who was previously diagnosed by a physician who instructs the primary care physician when the primary care physician for the diagnosis target patient is an inexperienced physician.
(Condition 3): The other patient is another patient who visited a clinical department to which the primary care physician for the diagnosis target patient belongs.
(Condition 4): The other patient is another patient for whom diagnosis results identical to the current diagnosis results for the diagnosis target patient were obtained.
(Condition 5): The other patient is another patient for whom diagnosis results close to the current diagnosis results for the diagnosis target patient were obtained (for example, another patient who was diagnosed with the same type of disease). Here, a diagnosis result close to the current diagnosis result for the diagnosis target patient may be determined using, for example, existing disease ontology technology.
(Condition 6): The other patient is another patient who was suspected of having a disease identical to or similar to the current diagnosis result for the diagnosis target patient, but who was ultimately diagnosed as having no problem (no abnormality).

The comparison target patient identification function 142 is performed to notify each of the feature selection function 144 and the reference data generation function 146 of information indicating past clinical data (hereinafter referred to as “specific past clinical data”) of other patients (comparison target patients) identified (decided on) from the past clinical data acquired by the clinical data acquisition function 120. The comparison target patient identification function 142 may be performed to designate the past clinical data as the specific past clinical data by adding information indicating an identified (decided) comparison target patient to the past clinical data acquired by the clinical data acquisition function 120 and output the specific past clinical data to the feature selection function 144 and the reference data generation function 146.

The comparison target patient identification function 142 is an example of a “comparison target identifier.”

The feature selection function 144 is performed to select a feature in the patient-specific clinical data and the specific past clinical data of the comparison target patient identified by the comparison target patient identification function 142. The feature selected in the feature selection function 144 is performed to indicate feature data to be referred to in the diagnosis procedure necessary to obtain a consistent diagnosis result for each patient. It is possible to determine whether or not the diagnosis procedure necessary to obtain the consistent diagnosis result has been performed, for example, according to states of reference (confirmation) for data such as the presence or absence of reference of data such as examination results, examination images, and medical interview results (diagnosis result data itself may also be included), types of data that has been referred to (classifications of examination results, examination images, medical interview results, and the like), and a period of time for which the data was referred to.

Thus, the feature selection function 144 is first performed to confirm a state of reference by a physician and a diagnosis support model for the patient-specific clinical data and the specific past clinical data and extract data that has been referred to as data of a candidate for a feature (hereinafter referred to as a “feature candidate”) to be selected. A process of confirming the state of reference for the patient-specific clinical data and the specific past clinical data in the feature selection function 144 is performed, for example, on the basis of the patient's electronic medical chart (a treatment storage device (not shown) in the electronic medical chart system), a storage device (not shown) storing patient examination results, a record (so-called operation log) of an operation performed by the physician for the clinical decision support system, and the presence and absence of reference (read or write access) based on the diagnosis support model.

More specifically, the feature selection function 144 is performed to confirm whether or not the primary care physician has personally and actually referred to (confirmed) data such as examination results, examination images, and medical interview results included in the patient-specific clinical data from the time the primary care physician started diagnosing the diagnosis target patient to the present time and a period of time for which the data was actually referred to and extract the data referred to by the primary care physician as the feature candidate. Likewise, the feature selection function 144 is also performed to confirm whether or not the primary care physician for the comparison target patient has personally and actually referred to data such as examination results, examination images, and medical interview results included in the specific past clinical data and the period of time for which the data was actually referred to and extract data referred to by the primary care physician for the comparison target patient as a feature candidate.

Furthermore, for example, the feature selection function 144 is performed to confirm whether or not data such as examination results, examination images, medical interview results, and the like included in the patient-specific clinical data has been referred to by the diagnosis support model and extract data referred to by the diagnosis support model as a feature candidate. Likewise, the feature selection function 144 is also performed to confirm whether or not data such as examination results, examination images, medical interview results, and the like included in the specific past clinical data has been referred to and extract data referred to by the diagnosis support model as a feature candidate. At this time, a case where the examination result data included in the specific past clinical data includes data capable of being made equivalent to the examination result data referred to by the diagnosis support model, for example, by performing a predetermined complementary process, is also conceivable. In this case, the feature selection function 144 may be performed to designate each examination result for which the complementary process is performed as data of an examination result referred to by the diagnosis support model in the specific past clinical data and output it as a feature candidate. For example, when data of biomarker examination results for heart failure such as left ventricular ejection fraction (LVEF) and N-terminal pro-brain natriuretic peptide (NT-proBNP) regarded as having a strong relationship (correlation) with the characteristic of the risk of heart failure output from the diagnosis support model are included in the specific past clinical data, data about the risk of heart failure based on the left ventricular ejection fraction and the human brain natriuretic peptide precursor N-terminal fragment may be extracted as feature candidates in the specific past clinical data.

The feature selection function 144 may be performed to extract, as feature candidates, all data referred to by a physician or a diagnosis support model from the patient-specific clinical data or the specific past clinical data. However, for example, in a case where data that does not need to be extracted as a feature candidate is set in advance by the physician or the like, the set data may not be extracted as a feature candidate.

In the following description, it is assumed that the types of data referred to by the physician and the diagnosis support model are extracted as feature candidates. In the following description, a feature candidate (type of data) personally and actually referred to by the physician, i.e., directly confirmed by the physician, is referred to as a “direct reference data type Dd” and a feature candidate (type of data) referred to by the diagnosis support model, i.e., indirectly confirmed by the physician, is referred to as an “indirect reference data type Di.” The direct reference data type Dd and the indirect reference data type Di are examples of “feature data.”

Subsequently, the feature selection function 144 is performed to select a feature in each item of the patient-specific clinical data and the specific past clinical data on the basis of the direct reference data type Dd and the indirect reference data type Di that have been extracted. At this time, the feature selection function 144 is performed to select a feature in each item of the patient-specific clinical data and the specific past clinical data in, for example, one of the following methods.
(Method a): Only the direct reference data type Dd is selected as a feature.
(Method b): Both the direct reference data type Dd and the indirect reference data type Di are selected as features. In other words, a logical sum of the direct reference data type Dd and the indirect reference data type Di is selected as a feature.
(Method c): Only the same feature (i.e., the same data type) is selected as a feature between the direct reference data type Dd and the indirect reference data type Di. In other words, a logical product of the direct reference data type Dd and the indirect reference data type Di is selected as a feature.
(Method d): A direct reference data type Dd and an indirect reference data type Di capable of being made equivalent to the direct reference data type Dd by performing a predetermined complementary process are selected as features. That is, a part of the indirect reference data type Di is included in the direct reference data type Dd and selected as a feature.

The feature selection function 144 is performed to notify the reference data generation function 146 of information indicating the selected feature. More specifically, the feature selection function 144 is performed to notify the reference data generation function 146 of information indicating the type of data selected from the specific past clinical data and information indicating the type of data selected from the patient-specific clinical data.

The feature selection function 144 is an example of a “feature selector.”

The reference data generation function 146 is performed to generate reference data corresponding to each patient associated as a feature quantity indicating a feature selected in the feature selection function 144. More specifically, for example, the reference data generation function 146 is performed to assign reference information indicating whether or not a feature has been selected in the feature selection function 144, i.e., whether data has been referred to by the physician or the diagnosis support model, as a feature quantity to the reference data for each patient whose data type has been set in advance. When the feature selected in the feature selection function 144 is the type of data referred to by the physician or the diagnosis support model, the reference information (feature quantity) is, for example, flag information indicating whether or not the feature has been referred to by the physician or the diagnosis support model. The reference data generation function 146 may be performed to assign flag information that can be distinguished from flag information indicating whether or not the data has been referred to by the physician and flag information indicating whether or not the data has been referred to by the diagnosis support model to the reference data. In other words, the reference data generation function 146 may be performed to assign flag information for distinguishing the direct reference data type Dd and the indirect reference data type Di selected in the feature selection function 144 to the reference data. The reference data generation function 146 may be performed to generate reference data of the past reference data pRef corresponding to the past clinical data and the patient reference data cRef corresponding to the patient-specific clinical data.

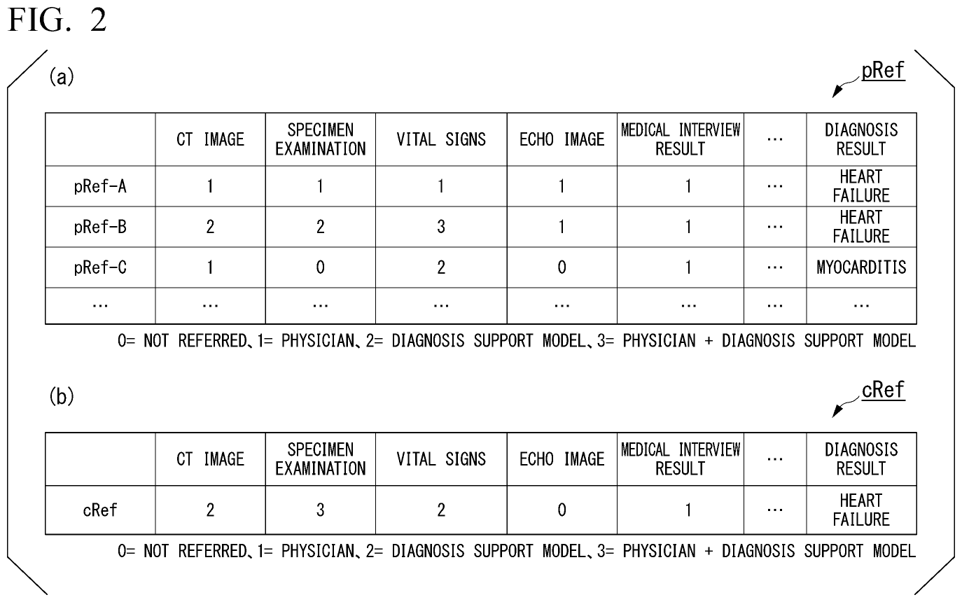

FIG. 2 is a diagram showing an example of information of reference data acquired by the medical diagnosis support device 100 according to the embodiment (more specifically, generated in the reference data generation function 146 of the reference data acquisition function 140). In FIG. 2(a), an example of the past reference data pRef generated by the reference data generation function 146 is shown. In FIG. 2(b), an example of the patient reference data cRef generated by the reference data generation function 146 is shown. More specifically, three items of past reference data pRef, i.e., past reference data pRef-A corresponding to comparison target patient A, past reference data pRef-B corresponding to comparison target patient B, and a past reference data pRef-C corresponding to comparison target patient C, are shown in FIG. 2(a) and patient reference data cRef corresponding to a current diagnosis target patient is shown in FIG. 2(b).

As shown in FIGS. 2(a) and 2(b), the same data type is set in advance for the past reference data pRef and the patient reference data cRef. More specifically, in reference data shown in FIGS. 2(a) and 2(b), data types such as “computed tomography (CT) image,” “specimen examination,” “vital signs,” “echo image,” “medical interview result,” and “diagnosis result” are set in advance. The reference data generation function 146 is performed to associate each data type in reference data with flag information indicating whether or not the data has been referred to by the physician or the diagnosis support model. In the past reference data pRef and the patient reference data cRef, the flag information=“0” indicates a type of data not referred to by either of the physician and the diagnosis support model, i.e., a type of data not included in either of the direct reference data type Dd and the indirect reference data type Di. On the other hand, in the past reference data pRef and the patient reference data cRef, the flag information=“1” indicates a type of data referred to only by the physician, i.e., a type of data included only in the direct reference data type Dd, and the flag information=“2” indicates a type of data referred to only by the diagnosis support model, i.e., a type of data included only in the indirect reference data type Di. In the past reference data pRef and the patient reference data cRef, the flag information=“3” indicates the type of data referred to by both the physician and the diagnosis support model, i.e., the type of data included in the direct reference data type Dd and the indirect reference data type Di. Past reference data pRef-A shown in FIG. 2(a) is an example in which “CT image,” “specimen examination,” “vital signs,” “echo image,” and “medical interview result” are referred to by the physician and “heart failure” is obtained as a diagnosis result. The past reference data pRef-A is an example of reference data that is not associated with the flag information referred to by the diagnosis support model, i.e., reference data obtained by diagnosing the comparison target patient before the introduction of the clinical decision support system. Past reference data pRef-B shown in FIG. 2(a) is an example in which “CT image” and “specimen examination” are referred to by the diagnosis support model, “vital signs” are referred to by both the physician and the diagnosis support model, “echo image” and “medical interview result” are referred to by the physician, and “heart failure” is obtained as the diagnosis result. Past reference data pRef-C shown in FIG. 2(a) is an example in which “CT image” and “medical interview result” are referred to by the physician, “vital signs” are referred to by the diagnosis support model, “specimen examination” and “echo image” are not referred to by either of the physician and the diagnosis support model, and “myocarditis” is obtained as the diagnosis result. The past reference data pRef-B and the past reference data pRef-C are examples of reference data for diagnosing a comparison target patient after the introduction of the clinical decision support system associated with the flag information referred to by the diagnosis support model.

The patient reference data cRef shown in FIG. 2(b) is an example in which “CT image” and “vital signs” are referred to by the diagnosis support model, “specimen examination” is referred to by both the physician and the diagnosis support model, “medical interview result” is referred to by the physician, “echo image” is not referred to by either of the physician and the diagnosis support model, and “heart failure” is obtained as the diagnosis result.

In this way, the reference data generation function 146 is performed to generate reference data for each patient associated with flag information indicating whether or not data has been referred to by the physician or the diagnosis support model. Here, it is considered that flag information=“0” to “3” in the reference data shown in FIGS. 2(a) and 2(b) can be implemented by expressing the flag information as 2-bit information. However, for example, if the reference information (feature quantity) about either of the physician and the diagnosis support model referred to data is unnecessary, “0” may denote flag information indicating that data has not been referred to by either of the physician and the diagnosis support model and “1” may denote flag information indicating that data has been referred to by one or both of the physician and the diagnosis support model.

Although an example of reference data when flag information indicating whether or not data has been referred to by the physician or the diagnosis support model is associated as reference information (a feature quantity) is shown in FIGS. 2(a) and 2(b), the reference information associated with the reference data may be associated with other information as the reference information in addition to or instead of the flag information indicating whether or not the data has been referred to. For example, the feature selection function 144 may be performed to associate information about a period of time for which the physician or diagnosis support model referred to the data as reference information. At this time, the feature selection function 144 may be performed to designate the period of time for which the data was referred to by the diagnosis support model as a valid period of time (for example, 5 [sec], 10 [sec], or the like) that is considered to be usually required for the physician to refer to (confirm) the data and make the determination. Here, when the data is data referred to by both the physician and the diagnosis support model and, for example, when the diagnosis support model referred to the data for 5 [sec] and the physician referred to the data for 10 [sec], the feature selection function 144 may be performed to associate 10 [sec] as the period of time for which data was referred to by prioritizing the period of time for which data was referred to by the physician. For example, the feature selection function 144 may be performed to associate, as reference information, information about a timing when the data was referred to by the physician or the diagnosis support model (for example, elapsed time from the time when the diagnosis of the patient started to the time when the data was referred to or the like).

The reference data generation function 146 is performed to output the generated past reference data pRef and the generated patient reference data cRef to the analysis function 160. For example, the reference data generation function 146 may be performed to control a communicator (not shown) so that the generated reference data is stored in the clinical database 10, another database (not shown), or a storage device (not shown) included in the medical diagnosis support device 100 and a notification indicating that the generated reference data has been stored is provided to the analysis function 160. When the generated reference data (in particular, the past reference data pRef) is stored in a storage device (not shown) or the like, the reference data generation function 146 can be performed to designate the past reference data pRef that has been stored as the past reference data pRef that has been generated without generating the same past reference data pRef, for example, at the time of the next diagnosis of the same diagnosis target patient, or at the time of diagnosis of another diagnosis target patient having the same diagnosis result as the diagnosis target patient. In this case, the processing load of the reference data generation function 146 to generate the past reference data pRef can be reduced.

The reference data generation function 146 is an example of a “reference data generator.”

The analysis function 160 is performed to perform an analysis process for the diagnosis result (which may be a diagnosis result indicated in the patient reference data cRef) for the diagnosis target patient indicated in the patient-specific clinical data on the basis of the past reference data pRef and the patient reference data cRef output in the reference data acquisition function 140 (more specifically, the reference data generation function 146). More specifically, the analysis function 160 is performed to analyze whether or not the trend of the feature quantity included in the patient reference data cRef matches the trend of the feature quantity included in the past reference data pRef, i.e., to perform an analysis process relating to a degree of matching of the trends of the feature quantities. For example, the analysis function 160 is performed to analyze whether or not the trend of the feature quantity included in the patient reference data cRef is similar to the trend of the feature quantity included in the past reference data pRef by performing a clustering process using a method of clustering the past reference data pRef and the patient reference data cRef as an unsupervised learning method. In this regard, when there is only one item of past reference data pRef to be analyzed, for example, when the medical diagnosis support device 100 is initially introduced, when only one comparison target patient to be compared with the diagnosis target patient is identified, or the like, a process of analyzing a degree of matching between the trend of the feature quantity included in the patient reference data cRef and the trend of the feature quantity included in the past reference data pRef may be performed by performing a distance calculation process instead of a clustering process. Here, because the feature selected in the feature selection function 144 is a type of data referred to by the physician or the diagnosis support model, the analysis function 160 may be performed to analyze whether or not a type of data associated with reference information (flag information) indicating that data has been referred to in the past reference data pRef is the same as a type of data associated with reference information (flag information) indicating that data has been referred to in the patient reference data cRef. The analysis function 160 is performed to evaluate whether or not the primary care physician has performed a consistent diagnosis procedure to obtain a diagnosis result for the diagnosis target patient on the basis of a result of an analysis process.

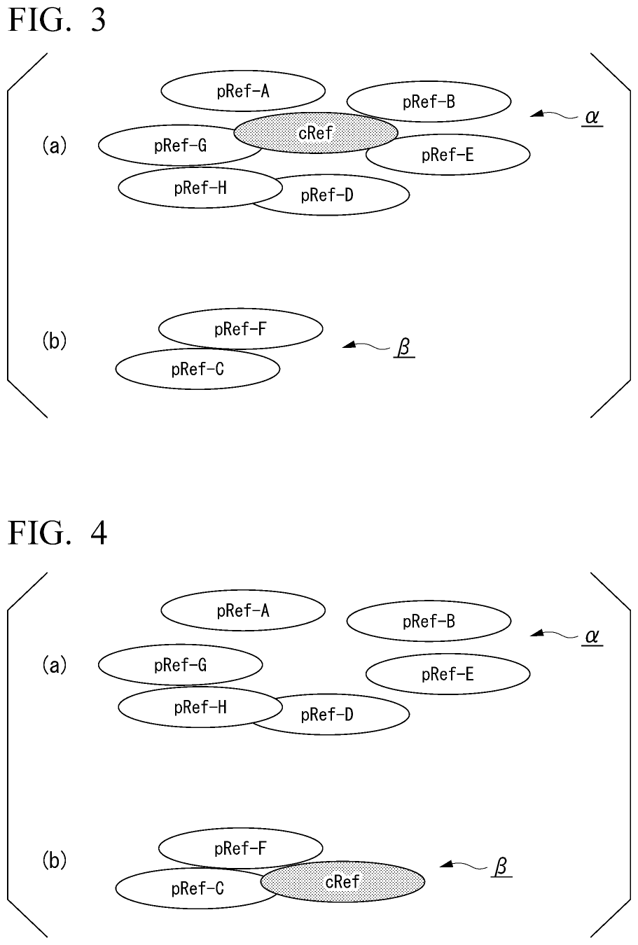

FIGS. 3 and 4 are diagrams schematically showing an example of a process in which the medical diagnosis support device 100 (more specifically, the analysis function 160) according to the embodiment analyzes a diagnosis result. In FIGS. 3 and 4, an example in which a plurality of items of the past reference data pRef (past reference data pRef-A to pRef-H) and patient reference data cRef are classified (clustered) into two classes is shown.

FIG. 3 is an example of a case where patient reference data cRef are classified into classes containing much past reference data pRef. More specifically, an example in which the patient reference data cRef is classified as class α between class α including much past reference data pRef shown in FIG. 3(a) and class β including little past reference data pRef shown in FIG. 3(b) is shown. In this case, a diagnosis result is obtained in a state in which the trend of the feature quantity included in the patient reference data cRef is similar to the trend of the feature quantity included in the past reference data pRef for the previous comparison target patient. Thereby, the analysis function 160 is performed to evaluate that the diagnosis result of the primary care physician for the current diagnosis target patient is obtained by performing the necessary diagnosis procedure.

FIG. 4 is an example in which patient reference data cRef is classified as a class containing little past reference data pRef. More specifically, an example in which the patient reference data cRef is classified as class β between class α including much past reference data pRef shown in FIG. 4(a) and class β including little past reference data pRef shown in FIG. 4(b) is shown. In this case, a diagnosis result is obtained in a state in which the trend of the feature quantity included in the patient reference data cRef is different from the trend of the feature quantity included in the past reference data pRef for the previous comparison target patient. Thereby, the analysis function 160 is performed to evaluate that the diagnosis result of the primary care physician for the current diagnosis target patient is obtained without performing the necessary diagnosis procedure.

The analysis function 160 is performed to output information indicating a result of performing an analysis process for the patient reference data cRef, i.e., a result of evaluating whether or not the primary care physician has performed a consistent diagnosis procedure to obtain a diagnosis result for the diagnosis target patient, to the result notification function 180. More specifically, when it is evaluated that the diagnosis result for the diagnosis target patient has been obtained by performing the necessary diagnosis procedure, the analysis function 160 is performed to output information indicating that the evaluation result is satisfactory to the result notification function 180. On the other hand, when it is evaluated that the diagnosis result for the diagnosis target patient was obtained without performing the necessary diagnosis procedures, the analysis function 160 is performed to output information indicating that the evaluation result has a problem and information indicating a difference between the trend of the feature quantity included in the much past reference data pRef classified as class α and the trend of the feature quantity included in the patient reference data cRef classified as class β to the result notification function 180. For example, the analysis function 160 is performed to output information indicating a type of data not associated with similar flag information in the patient reference data cRef (i.e., information indicating an examination result, an examination image, a medical interview result, and the like) to the result notification function 180 even through flag information indicating that data has been referred to in all past reference data pRef classified as class α by the physician or the diagnosis support model is associated.

The analysis function 160 is an example of an “analyzer.”

Although a case where it is evaluated whether or not a diagnosis result is a diagnosis result of performing a consistent diagnosis procedure by performing an analysis process relating to a degree of matching between the trend of the feature quantity included in the patient reference data cRef and the trend of the feature quantity included in the past reference data pRef in the analysis function 160 described above has been described, this is merely an example. The analysis function 160 may be performed to evaluate (determine) whether or not the consistent diagnosis procedure has been performed, for example, by quantifying a likelihood that a consistent diagnosis procedure has been performed on the basis of the size of each classified class and determining the quantized value on the basis of a predetermined threshold value or the like.

More specifically, for example, if a class including patient reference data cRef is designated as class A and a class not including patient reference data cRef is designated as class B, the analysis function 160 is performed to obtain a likelihood P that the consistent diagnosis procedure has been performed according to the following Eq. (1).

P=size of class A/(size of class A+size of class B) (1)

Also, the analysis function 160 may be performed to evaluate that the diagnosis result of the primary care physician for the current diagnosis target patient is obtained by performing a consistent diagnosis procedure when the obtained likelihood P is greater than the predetermined threshold value and evaluate that the diagnosis result of the primary care physician for the current diagnosis target patient is obtained without performing a consistent diagnosis procedure when the obtained likelihood P is less than or equal to the predetermined threshold value.

Although a case where it is evaluated whether or not the diagnosis result is a diagnosis result of performing a consistent diagnosis procedure using the clustering method, which is an unsupervised learning method, in the above-described analysis function 160, has been described, this is also merely an example. The analysis function 160 may be performed to evaluate (determine) whether or not the consistent diagnosis procedure has been performed using, for example, a supervised learning method. In this case, for example, a trained model that has learned a degree of matching (a matching degree) of the trend of the feature quantity included in the reference data using an artificial intelligence (AI) function (hereinafter referred to as a “matching-degree-related trained model”) may be provided in advance for each of methods (methods a to d) of selecting a feature in the above-described feature selection function 144. The matching-degree-related trained model is, for example, a trained model trained to output the degree of matching between trends of feature quantities shown in input reference data using machine learning technology such as a convolutional neural network (CNN) or a deep neural network (DNN) as a determination result. Here, the CNN is a neural network in which several layers such as a convolution layer and a pooling layer are connected. The DNN is a neural network in which layers of any form are connected in multiple layers. The matching-degree-related trained model may be generated in machine learning using a machine learning model by, for example, a computing device (not shown). In this case, past reference data pRef based on the past clinical data of a previously diagnosed patient or the like is input as input data to the input side of the matching-degree-related trained model in a calculation device (not shown) when the matching-degree-related trained model is generated and a diagnosis result of a diagnosis process performed by a primary care physician, an experienced physician, or the like, data referred to thereby, or the like may be input as training data to an output side of the matching-degree-related trained model.

The result notification function 180 is performed to generate notification information for notifying the primary care physician of the evaluation result on the basis of the information indicating the evaluation result output in the analysis function 160. For example, the result notification function 180 is performed to notify the primary care physician of the evaluation result output in the analysis function 160 by generating a display image for displaying the display content indicating the evaluation result and causing a display device (not shown) connected to the medical diagnosis support device 100 to display the generated display image. The result notification function 180 may be performed to notify the primary care physician of the evaluation result output in the analysis function 160, for example, by controlling a communicator (not shown) so that the communicator (not shown) is allowed to transmit a display image generated by a terminal device connected to the network NW and used when the primary care physician diagnoses the diagnosis target patient, a terminal device for executing a function of the clinical decision support system, or the like and a display device provided in the terminal device or the display device connected to the terminal device is allowed to display the display image.

The result notification function 180 is an example of a “display controller.”

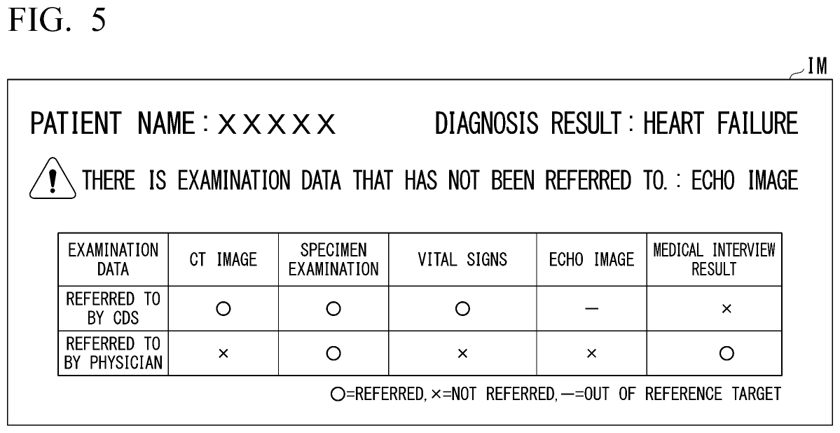

FIG. 5 is a diagram showing an example of a method in which the medical diagnosis support device 100 according to the embodiment provides a notification of a result of analyzing the diagnosis result. In FIG. 5, an example of a display screen IM on which the display content indicating the evaluation result is displayed on the display device is shown. The display screen IM shown in FIG. 5 shows an example in which a notification indicating that the primary care physician has not referred to the “echo image” is provided when a diagnosis result of “heart failure” has been obtained in the patient reference data cRef shown in FIG. 2(b). Thereby, the primary care physician can recognize that the diagnosis in which the echo image has been referred to as a diagnosis procedure necessary to obtain the diagnosis result has not been performed.

Although an example of a case in which a notification indicating that the primary care physician has not referred to the “echo image” is provided by text in the display screen IM shown in FIG. 5 is shown, this is merely an example and a method of providing a notification of examination data that has not been referred to by the primary care physician may be another method. For example, the result notification function 180 may be performed to change the color of an “echo image” item that has not been referred to by the primary care physician to highlight it or display text in a balloon in an examination data table shown on the display screen IM. For example, the result notification function 180 may be performed to display icons for distinguishing “confirmed by physician,” “confirmed by clinical decision support system,” “confirmed by physician and clinical decision support system,” and the like in the examination data table displayed on display screen IM. For example, the result notification function 180 may be performed to display a check box for examination data that has not been referred to and input a checkmark in the check box after confirming the examination data whose notification has been received. As described above, the result notification function 180 may use any method of notifying the primary care physician that the examination data has not been referred to through the display screen as long as the method makes it easy for the primary care physician to recognize.

### [Process of Medical Diagnosis Support Device]

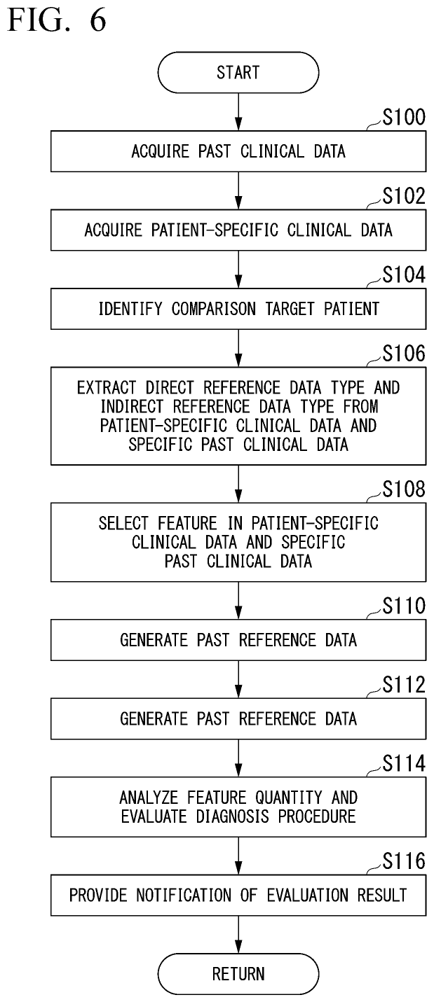

Next, an operation of the medical diagnosis support device 100 will be described. FIG. 6 is a flowchart showing an example of a flow of a process in the medical diagnosis support device 100 according to the embodiment. In FIG. 6, an example of a process in which the medical diagnosis support device 100 evaluates whether or not the primary care physician has performed a consistent diagnosis procedure after the primary care physician has ended a process of diagnosing a diagnosis target patient is shown. After the diagnosis target patient is diagnosed, the medical diagnosis support device 100 may start a process of evaluating a diagnosis procedure in accordance with an instruction from the primary care physician.

When the medical diagnosis support device 100 (the processing circuitry 110) starts a process of evaluating a diagnosis procedure, the clinical data acquisition function 120 is performed to acquire past clinical data stored in the clinical database 10 (step S100). Furthermore, the clinical data acquisition function 120 acquires patient-specific clinical data stored in the clinical database 10 (step S102). The clinical data acquisition function 120 is performed to output the acquired past clinical data and the acquired patient-specific clinical data to the reference data acquisition function 140.

A comparison target patient identification function 142 of the reference data acquisition function 140 is performed to identify a comparison target patient to be compared with a diagnosis target patient indicated in the patient-specific clinical data from other patients indicated in the past clinical data acquired in the clinical data acquisition function 120 (step S104). The comparison target patient identification function 142 is performed to notify the feature selection function 144 and the reference data generation function 146 of information indicating the past clinical data (specific past clinical data) of the identified (determined) comparison target patient.

The feature selection function 144 of the reference data acquisition function 140 is performed to extract a direct reference data type Dd and an indirect reference data type Di from the patient-specific clinical data acquired in the clinical data acquisition function 120 and the specific past clinical data of the comparison target patient identified in the comparison target patient identification function 142 (step S106). Also, the feature selection function 144 is performed to select a feature in the patient-specific clinical data and the specific past clinical data on the basis of the extracted direct reference data type Dd and the extracted indirect reference data type Di (step S108). The feature selection function 144 is performed to notify the reference data generation function 146 of information indicating the selected feature.

The reference data generation function 146 of the reference data acquisition function 140 is performed to generate past reference data pRef corresponding to the specific past clinical data acquired in the clinical data acquisition function 120 on the basis of the information indicating the feature selected in the feature selection function 144 (step S110). Furthermore, the reference data generation function 146 is performed to generate patient reference data cRef corresponding to the patient-specific clinical data acquired in the clinical data acquisition function 120 on the basis of information indicating the feature selected in the feature selection function 144 (step S112). The reference data generation function 146 is performed to output reference data of each item of the generated past reference data pRef and the generated patient reference data cRef to the analysis function 160.

The analysis function 160 is performed to perform a process of analyzing the feature quantity in the patient reference data cRef on the basis of the past reference data pRef and the patient reference data cRef output in the reference data generation function 146 and evaluate a diagnosis procedure performed by the primary care physician to obtain the diagnosis result for the diagnosis target patient on the basis of a result of the analysis process (step S114). The analysis function 160 is performed to output information indicating a result of evaluating the diagnosis procedure performed by the primary care physician to the result notification function 180.

The result notification function 180 is performed to generate notification information (for example, the display screen IM shown in FIG. 5) based on the information indicating the evaluation result output by the analysis function 160 and provide the generated notification (step S116). Also, the medical diagnosis support device 100 (the processing circuitry 110) ends the process of the present flowchart.

In this way, the medical diagnosis support device 100 can evaluate whether or not the consistent diagnosis procedure has been performed by the primary care physician for the diagnosis target patient to obtain the diagnosis result on the basis of the past clinical data and the patient-specific clinical data stored in the clinical database 10. As described above, the medical diagnosis support device of the embodiment generates patient reference data based on the patient-specific clinical data and specific past clinical data of the comparison target patient corresponding to the diagnosis target patient indicated in the patient-specific clinical data stored in the clinical database. Also, the medical diagnosis support device of the embodiment analyzes a feature quantity in the generated patient reference data on the basis of the generated past reference data. The feature quantity at this time indicates a feature including the data referred to by the diagnosis support model used in the function of the clinical decision support system as well as the data referred to by the physician. In other words, the feature quantity indicates a feature based on the viewpoints of both the physician and the diagnosis support model. Thereby, in the medical diagnosis support device of the embodiment, it is possible to evaluate whether or not the primary care physician for the diagnosis target patient has performed a consistent diagnosis procedure to obtain the diagnosis result. In the medical diagnosis support device of the embodiment, the primary care physician for the diagnosis target patient can recognize insufficient confirmation examination data to obtain the diagnosis result and a change or a change point in the procedure performed to obtain the diagnosis result. Thereby, a medical institution where the medical diagnosis support device of the embodiment is introduced can maintain the quality of a diagnosis procedure for a diagnosis target patient and obtain a consistent and more appropriate diagnosis result.

In the above-described embodiment, a case where the medical diagnosis support device 100 starts a process of evaluating the diagnosis procedure after the end of the diagnosis of the diagnosis target patient has been described. However, a timing at which the medical diagnosis support device 100 starts the process of evaluating the diagnosis procedure may be any timing according to an instruction from the primary care physician who is diagnosing the diagnosis target patient. In this case, it is only necessary for the reference data acquisition function 140 to be performed to generate patient reference data cRef based on current patient-specific clinical data (which does not necessarily include a diagnosis result) and perform a process of evaluating a diagnosis procedure. Thereby, the primary care physician for the diagnosis target patient can identify whether or not his/her diagnosis procedure is appropriate at any timing. It is only necessary for the functional configuration, operation, process, and the like of the medical diagnosis support device 100 in this case to be equivalent to the functional configuration, operation, process, and the like of the medical diagnosis support device 100 of the above-described embodiment.

In the above-described embodiment, a case where the reference data of the past reference data pRef and the patient reference data cRef is generated after the start of the process of evaluating the diagnosis procedure has been described. However, a case where the generated reference data (particularly, past reference data pRef) need not be generated each time a process of evaluating the diagnosis procedure is performed is also conceivable. This is because the past reference data pRef is reference data based on past clinical data and is not considered to be updated each time a diagnosis target patient is diagnosed like patient-specific clinical data. Therefore, the processing load for generating the same past reference data pRef can be reduced by storing the generated past reference data pRef in a storage device (not shown) or the like as described above. The functional configuration, operation, process, and the like of the medical diagnosis support device 100 in this case can be easily conceivable on the basis of the functional configuration, operation, process, and the like of the medical diagnosis support device 100 of the above-described embodiment. Accordingly, detailed descriptions of the functional configuration, operation, process, and the like of the medical diagnosis support device 100 in this case will be omitted.

The embodiment described above can be represented as follows.

A medical diagnosis support device including:
processing circuitry configured to
acquire first reference data based on first clinical data relating to a diagnosis result of an evaluation target and second reference data based on second clinical data relating to a comparison target diagnosis result for the evaluation target and perform an analysis process relating to the diagnosis result of the evaluation target on the basis of the first reference data and the second reference data,
wherein the first reference data includes first data that has been referred to by a first user to obtain the diagnosis result of the evaluation target and second data that has been referred to by a diagnosis support system to obtain the diagnosis result of the evaluation target, and
wherein the second reference data includes the first data that has been referred to by at least one of the first user and a second user different from the first user in order to obtain the diagnosis result of the comparison target.

According to at least one embodiment described above, there is provided processing circuitry configured to acquire first reference data (cRef) based on first clinical data relating to a diagnosis result of an evaluation target (a present patient) and second reference data (pRef) based on second clinical data relating to a comparison target (past patient) diagnosis result for the evaluation target (140) and perform an analysis process relating to the diagnosis result of the evaluation target on the basis of the first reference data and the second reference data (160), wherein the first reference data includes first data (Dd) that has been referred to by a first user (a primary care physician) to obtain the diagnosis result of the evaluation target and second data (Di) that has been referred to by a diagnosis support system to obtain the diagnosis result of the evaluation target, and wherein the second reference data includes the first data that has been referred to by at least one of the first user and a second user (a primary care physician for another patient) different from the first user in order to obtain the diagnosis result of the comparison target, whereby it is possible to support a diagnosis process of a first user while executing a procedure of the first user necessary to obtain a diagnosis result.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical diagnosis support device comprising:
processing circuitry configured to
acquire first reference data based on first clinical data relating to a diagnosis result of an evaluation target and second reference data based on second clinical data relating to a comparison target diagnosis result for the evaluation target and
perform an analysis process relating to the diagnosis result of the evaluation target based on the first reference data and the second reference data,
wherein the first reference data includes first data that has been referred to by a first user to obtain the diagnosis result of the evaluation target and second data that has been referred to by a diagnosis support system to obtain the diagnosis result of the evaluation target, and
wherein the second reference data includes the first data that has been referred to by at least one of the first user and a second user different from the first user in order to obtain the diagnosis result of the comparison target.

2. The medical diagnosis support device according to claim 1, wherein the processing circuitry
acquires a plurality of items of second reference data based on a plurality of items of second clinical data, and
performs the analysis process based on a clustering process including the first reference data and the plurality of items of second reference data.

3. The medical diagnosis support device according to claim 2, wherein the processing circuitry
identifies the second clinical data in which the comparison target for the evaluation target indicated in the first clinical data is indicated from among the plurality of items of second clinical data,
extracts feature candidates indicating a feature of data necessary to obtain the diagnosis result of the evaluation target in the first clinical data and feature candidates indicating a feature of data necessary to obtain the diagnosis result of the comparison target in the identified second clinical data to select at least the first data as feature data from among the extracted feature candidates,
generates the first reference data and the second reference data associated with reference information indicating whether or not the selected feature data has been referred to, and
performs an analysis process relating to a degree of matching between the first reference data and the second reference data based on the feature data associated with the reference information.

4. The medical diagnosis support device according to claim 3, wherein the processing circuitry identifies the second clinical data in which the comparison target having the same diagnosis result as the evaluation target is indicated.

5. The medical diagnosis support device according to claim 3, wherein the processing circuitry identifies the second clinical data in which the comparison target having a diagnosis result close to a diagnosis result of the evaluation target is indicated.

6. The medical diagnosis support device according to claim 3, wherein the processing circuitry selects the feature data including the second data.

7. The medical diagnosis support device according to claim 6, wherein the processing circuitry selects all the first data and all the second data as the feature data.

8. The medical diagnosis support device according to claim 6, wherein the processing circuitry selects the first data and the second data having the same data feature as the feature data from among all the first data and all the second data.

9. The medical diagnosis support device according to claim 6, wherein the processing circuitry selects all the first data and the second data complementary to the first data as the feature data.

10. The medical diagnosis support device according to claim 3, wherein the processing circuitry associates the reference information indicating whether or not the selected feature data has been referred to by the first user with the first reference data and the second reference data.

11. The medical diagnosis support device according to claim 10, wherein the processing circuitry associates the reference information indicating whether or not the selected feature data has been referred to by the diagnosis support system with the first reference data and the second reference data.

12. The medical diagnosis support device according to claim 1, wherein the processing circuitry causes a display device to display display content based on an analysis result of performing an analysis process relating to the diagnosis result of the evaluation target.

13. The medical diagnosis support device according to claim 12, wherein the processing circuitry causes the display device to display at least the display content indicating the second data to be referred to by the diagnosis support system without being referred to by the first user in the first reference data.

14. A medical diagnosis support method comprising:
acquiring, by a computer, first reference data based on first clinical data relating to a diagnosis result of an evaluation target and second reference data based on second clinical data relating to a comparison target diagnosis result for the evaluation target; and
performing, by the computer, an analysis process relating to the diagnosis result of the evaluation target based on the first reference data and the second reference data,
wherein the first reference data includes first data that has been referred to by a first user to obtain the diagnosis result of the evaluation target and second data that has been referred to by a diagnosis support system to obtain the diagnosis result of the evaluation target, and
wherein the second reference data includes the first data that has been referred to by at least one of the first user and a second user different from the first user in order to obtain the diagnosis result of the comparison target.

15. A storage medium for causing a computer to:
acquire first reference data based on first clinical data relating to a diagnosis result of an evaluation target and second reference data based on second clinical data relating to a comparison target diagnosis result for the evaluation target; and
perform an analysis process relating to the diagnosis result of the evaluation target based on the first reference data and the second reference data,
wherein the first reference data includes first data that has been referred to by a first user to obtain the diagnosis result of the evaluation target and second data that has been referred to by a diagnosis support system to obtain the diagnosis result of the evaluation target, and
wherein the second reference data includes the first data that has been referred to by at least one of the first user and a second user different from the first user in order to obtain the diagnosis result of the comparison target.
